(19) United States
(12) Patent Application Publication
(10) Pub. No.: US 2024/0042200 A1
(43) Pub. Date: Feb. 8, 2024

(54) Respiration Monitoring Sensor for a Laryngeal Pacemaker

(71) Applicant: MED-EL Elektromedizinische Geräte GmbH, Innsbruck (AT)

(72) Inventors: Daniele Santonocito, Innsbruck (AT); Alberto Lombardi, Innsbruck (AT); Rami Saba, Innsbruck (AT); Francesca Maule, Innsbruck (AT); Christian Diekow, Innsbruck (AT); Christian Denk, Innsbruck (AT); Christiane Poschl, Innsbruck (AT)

(21) Appl. No.: 18/378,379

(22) Filed: Oct. 10, 2023

Related U.S. Application Data
(63) Continuation of application No. 17/409,276, filed on Aug. 23, 2021, now abandoned.
Application No. 17/409,276 is a continuation of application No. 16/347,637, filed on May 6, 2019, now abandoned.
(60) Provisional application No. 62/426,647, filed on Nov. 28, 2016.

(51) Int. Cl. A61N 1/36; A61N 1/05

(52) U.S. Cl. CPC A61N 1/3601; A61N 1/0519; A61N 1/3611

(57) ABSTRACT

A laryngeal pacemaker is configured for external placement on skin of a patient to produce respiration stimulation signals. An implantable stimulation electrode delivers the respiration stimulation signals to adjacent target neural tissue for vocal fold abduction during respiration of the recipient patient. A triaxial accelerometer produces a body motion signal reflecting energy expenditure of the recipient patient. A respiration sensor includes a flexible skin-transferrable printed tattoo electrode having a tetrapolar configuration for impedance pneumography measurement to produce a sensed respiration signal for the laryngeal pacemaker. The respiration sensor is configured for transfer and release by guided placement from a sensor applicator to the skin at the angulus sterni of the recipient patient. And the laryngeal pacemaker is configured to interpret the body motion signal and the sensed respiration signal to make a real time determination of respiratory phase and frequency for adaptively adjusting the respiration stimulation signals accordingly.

### CROSS-REFERENCE TO RELATED APPLICATIONS

This application is a continuation of U.S. patent application Ser. No. 17/409,276, filed Aug. 23, 2021, which is a continuation of U.S. patent application Ser. No. 16/347,637 filed May 6, 2019, which is the national phase entry of International Patent Application No. PCT/US2017/063225 filed Nov. 27, 2017, which claims priority from U.S. Provisional Patent Application No. 62/426,647 filed Nov. 28, 2016, the disclosures of which are incorporated herein by reference in their entirety.

### TECHNICAL FIELD

The present invention relates to respiration sensors for laryngeal pacemaker systems.

### BACKGROUND ART

The larynx is located in the neck and is involved in breathing, producing sound (speech), and protecting the trachea from aspiration of food and water. FIG. 1A shows a coronal section view and FIG. 1B shows a transverse section view of the anatomy of a human larynx including the epiglottis 101, thyroid cartilage 102, vocal folds 103, cricothyroid muscle 104, arytenoid cartilage 105, posterior cricoarytenoid muscle (PCAM) 106, vocalis muscle 107, cricoid cartilage 108, recurrent laryngeal nerve (RLN) 109, transverse arytenoid muscle 110, oblique arytenoid muscle 111, superior laryngeal nerve 112, and hyoid bone 113.

The nerves and muscles of the larynx abduct (open) the vocal folds 103 during the inspiration phase of breathing to allow air to enter the lungs. And the nerves and muscles of the larynx adduct (close) the vocal folds 103 during the expiration phase of breathing to produce voiced sound. At rest, respiration frequency typically varies from 12 to 25 breaths per minute. So, for example, 20 breaths per minute result in a 3 second breath duration, with 1.5 sec inspiration, and 1.5 sec exhalation phase (assuming a 50/50 ratio). The breathing frequency changes depending on the physical activity.

Unilateral and bilateral injuries or ruptures of the recurrent laryngeal nerve (RLN) 109 initially result in a temporal partial paralysis of the supported muscles in the larynx (and the hypolarynx). A bilateral disruption of the RLN 109 causes a loss of the abductor function of both posterior cricoarytenoid muscles (PCAM) 106 with acute asphyxia and life-threatening conditions. This serious situation usually requires surgical treatment of the bilateral vocal cord paralysis such as cordotomy or arytenoidectomy, which subsequently restrict the voice and puts at risk the physiologic airway protection.

A more recent treatment approach to RLN injuries uses a laryngeal pacemaker that electrically stimulates (paces) the PCAM 106 during inspiration to abduct (open) the vocal folds 103. During expiration, the vocal folds 103 relax (close) to facilitate voicing. In first generation laryngeal pacemaker systems, the patient can vary the pacing frequency (breaths per minute) according to his physical load (at rest, normal walking, stairs, etc.) by manually switching the stimulation frequency of the pacer device, the assumption being that the human body may adapt to the artificial externally applied respiration frequency—within some locking-range. Thus the patient and the laryngeal pacemaker can be described as free running oscillators at almost the same frequency, but without phase-matching (no phase-locking). Sometimes both systems will be in phase, but other times the systems will be out of phase and thus the benefit for the patient will be reduced.

More recent second generation laryngeal pacemaker systems generate a stimulation trigger signal to synchronize the timing of stimulation of the pacemaker to the respiration cycle of the patient. The stimulation trigger signal defines a specific time point during the respiration cycle to initiate stimulation of the target neural tissue. The time point may specifically be the start or end of the inspiratory or expiratory phase of breathing, a breathing pause, or any other defined time point. To detect the desired time point, several types of respiration sensors have been investigated to generate a respiration sensing signal that varies within each breathing cycle. These include, for example, various microphones, accelerometer sensors, and pressure sensors (positioned in the pleura gap). Electromyogram (EMG) measurements also are under investigation for use in developing a stimulation trigger signal.

FIG. 2 shows one embodiment of such a laryngeal pacemaker system with a processor 201 that receives a respiration signal from a respiration sensor 202 implanted in the parasternal muscle that detects respiration activity in the implanted patient. Optionally, a three-axis acceleration movement sensor also is located within the housing of the processor 201 and generates a movement signal. Based on the respiration signal, the processor 201 generates a respiration pacing signal that is synchronized with the detected respiration activity and delivers the pacing signal via a processor lead to a stimulating electrode 203 implanted in the target respiration neural tissue to promote breathing of the implanted patient.

In conventional laryngeal pacemakers, many different kinds of respiration sensors have been proposed. Many such arrangements require connection to the pacing processor via an insulated conductive wire element embedded in a wire lead. Such a wire lead, though, may be prone to physical damage, requires effort for insertion during surgery, and has to somehow be securely fixed within delicate tissue, e.g. near or around nerves or within muscles.

### SUMMARY

Embodiments of the present invention are directed to a laryngeal pacing system for a recipient patient with impaired breathing. A laryngeal pacemaker is configured for external placement on skin of a patient at a sternum location and configured to produce respiration stimulation signals. An implantable stimulation electrode is configured for delivering the respiration stimulation signals from the laryngeal pacemaker to adjacent target neural tissue for vocal fold abduction during respiration of the recipient patient. A respiration sensor includes a flexible skin-transferrable printed tattoo electrode having a tetrapolar configuration for impedance pneumography measurement to produce a sensed respiration signal for the laryngeal pacemaker. A triaxial accelerometer is configured to produce a body motion signal for the laryngeal pacemaker that reflects energy expenditure of the recipient patient. The flexible skin-transferrable printed tattoo electrode (PTE) is configured for transfer and release by guided placement from a sensor applicator to a fixed skin location at the angulus sterni of the recipient patient. And the laryngeal pacemaker is configured to interpret the body motion signal and the sensed respiration signal to make a real time determination of respiratory phase and frequency for adaptively adjusting the respiration stimulation signals accordingly.

In a specific embodiment, the laryngeal pacemaker may include an outer surface with sensor contacts configured to directly connect to the PTE for coupling the sensed respiration signal from the respiration sensor to the laryngeal pacemaker. The triaxial accelerometer may be integrated into the respiration sensor. And the printed tattoo electrode may be formed from Tattoo Conductive Polymer Nanosheets for Skin-Contact Applications. The respiration sensor also may include a center support ring configured to mechanically engage the respiration sensor with the laryngeal pacemaker.

The respiration sensor may be configured for transfer and release using a water-based transfer mechanism. For example, there may be a semi-rigid support layer configured to provide mechanical support to the printed tattoo electrode and configured to release a wetting layer of water when mechanically pressed. Such a support layer may include multiple water-holding sub-divisions, or just a single water-holding sub-division. And the respiration sensor may be adapted to cooperate with the sensor applicator to provide positioning feedback information when the respiration sensor is placed at the fixed skin location.

### BRIEF DESCRIPTION OF THE DRAWINGS

FIG. 1A shows a coronal section view and FIG. 1B shows a transverse section view of the anatomy of a human larynx.

FIG. 2 shows a typical conventional laryngeal pacemaker arrangement with respect to patient anatomy.

FIG. 3 shows a laryngeal pacemaker arrangement with a respiration sensor according to an embodiment of the present invention.

FIGS. 4A-4D show various structural details associated with a respiration sensor arrangement according to an embodiment of the present invention.

FIG. 5 shows schematic details of a respiration sensor according to an embodiment of the present invention.

FIGS. 6A-6B show various aspects of the principle of IPG measurement according to an embodiment of the present invention.

FIGS. 7A-7B show various aspects of another specific embodiment of a respiration sensor with a support layer and water pockets according to an embodiment of the present invention.

FIG. 8 shows details of a support layer and water pocket according to another embodiment of the present invention.

FIG. 9 shows structural details of an alternative bottom surface of a laryngeal pacemaker.

FIG. 10 shows various waveforms associated with signal processing of a sensed respiration sensor according to an embodiment of the present invention.

FIGS. 11A-11B show various structural relationships in a sensor applicator according to an embodiment of the present invention.

FIG. 12 shows an exploded view of various structural elements of a sensor applicator according to an embodiment of the present invention.

FIGS. 13A-13D show various aspects in using a sensor applicator according to an embodiment of the present invention.

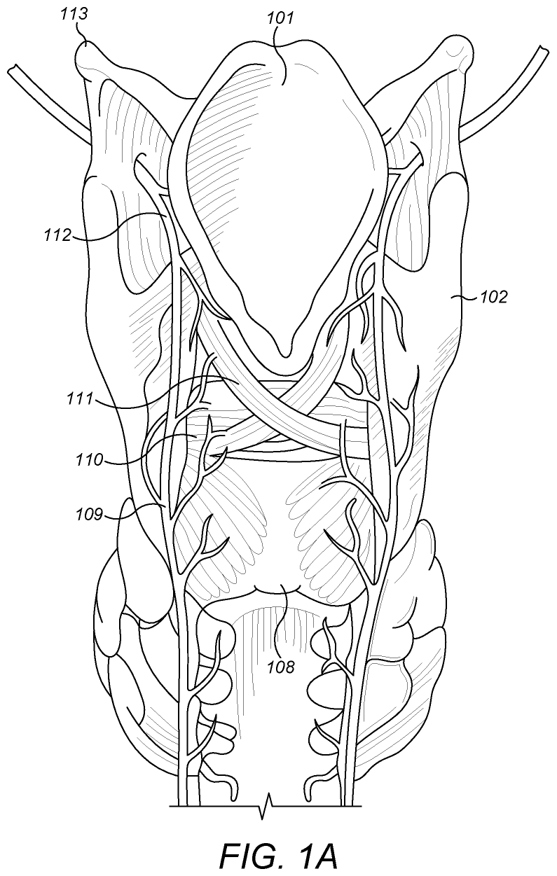

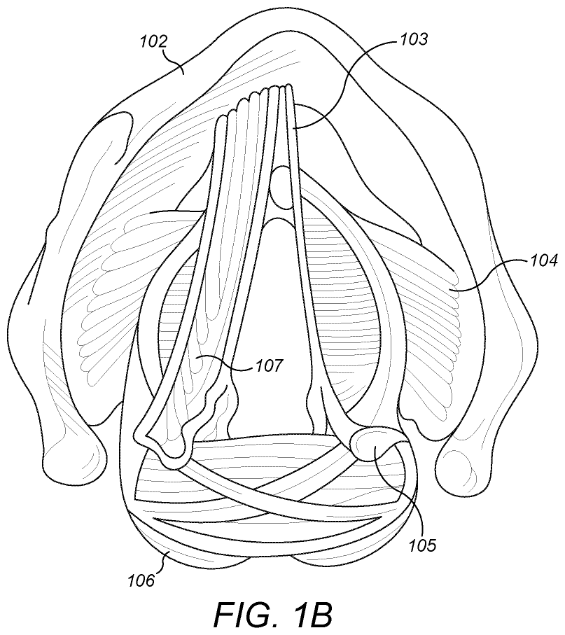

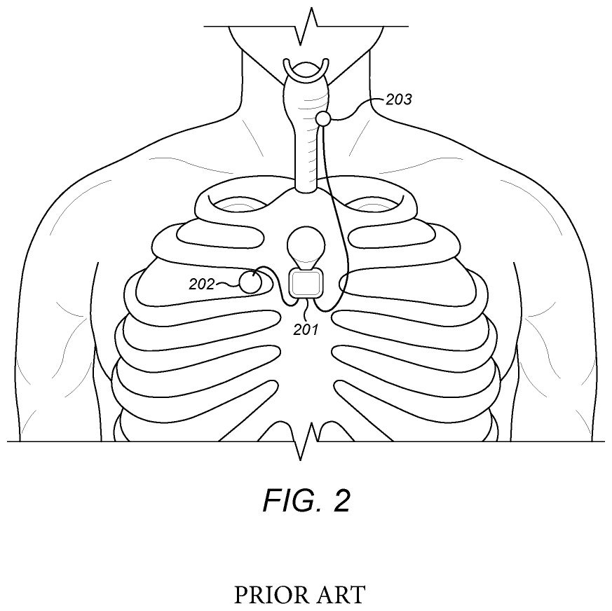

### DETAILED DESCRIPTION

Most existing respiration monitoring systems can only track the overall respiration rate (RR) over time, and not the true instantaneous respiration phase and frequency in real time. One reason for that is because the measured respiration signal is usually contaminated with a high amount of noise. One of the main sources of this noise in the measured respiratory signal is motion artifacts from physical movement of the sensor electrodes with respect to the tissue being measured. The motion artifacts are in the same frequency range as the respiration signal (0.1-1.0 Hz) and as a result cannot be easily filtered out. Consequently, it is difficult to derive a measurement signal that can reliably provide the instantaneous phase and frequency of the respiration.

Various embodiments of the present invention are directed to a laryngeal pacing system for a recipient patient with impaired breathing. RLN stimulation is triggered in phase with the true real-time respiration activity by using a respiration sensor that tracks the instantaneous respiration phase and frequency, communicates it to the electronics of the laryngeal pacemaker which adaptively adjusts the respiration stimulation signals accordingly. A respiration sensor is placed and coupled close to an external portion of the system, i.e. the processor device, which is easy to replace by other identical sensor means. In particular, patients can properly locate and attach the respiration sensor on their own without need of professional assistance. The respiration sensor of the present invention together with proper placement yields a very accurate estimation of the onset of the respiratory phase to which a stimulation signal has to be correlated. Unlike many prior art sensor systems, a particular phase of the respiratory cycle (e.g. the onset) can be reliably detected and not just the overall respiratory rate.

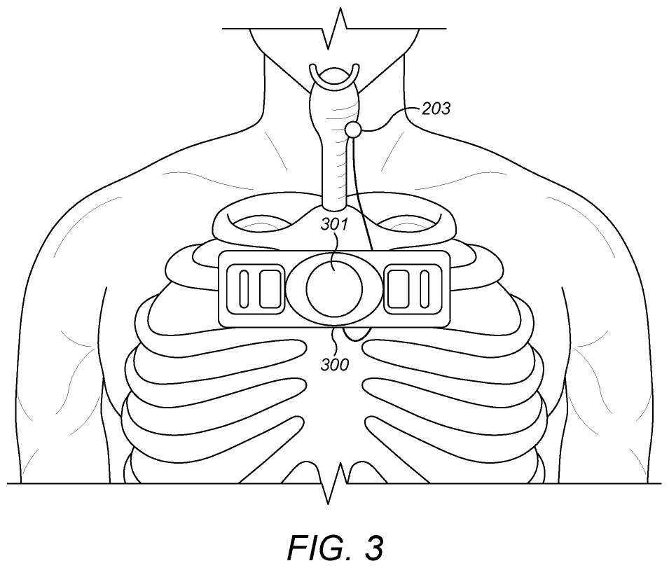

FIG. 3 shows an example of a laryngeal pacemaker arrangement with respect to patient anatomy according to an embodiment of the present invention which is configured to provide a reliable respiration signal that reflects real-time instantaneous respiration phase and frequency. A laryngeal pacemaker 301 is configured for external placement on the skin of the patient at as shown in FIG. 3 at the angulus sterni of the patient, and is configured to produce respiration stimulation signals. There is an internal implant portion of the system at that location which includes a holding magnet that cooperates with a corresponding holding magnet in the external laryngeal processor 301 to hold the latter in place. In addition, the angulus sterni location is advantageous because of the ideal flat shape of the bone there, and because there is a low percentage of fat in the underlying skin layer. The thickness of the underlying pectoralis muscle decreases dramatically at the angulus sterni, and the bone there is not part of any joint that might be subject to roll, pitch and yaw. This therefore represents a stable position during periods of high body movement. The width of the manubrium sterni depends on the sex of the subject, but an average width of 5.5 cm can be assumed. That means that changes in the lung impedance due to respiration can be easily measured at the manubrium sterni sides, if at least 5.5 cm distance is given between sensing electrodes located left and right of the angulus sterni. In short, the angulus sterni is an ideal placement for measurement of bio-impedance as discussed further below since motion induced artifacts are considerably reduced. We have identified this position as the best one for measuring reliably an inspiratory signal in the way described below.

As shown in FIG. 3, an implantable stimulation electrode 203 as known in the art is configured for delivering the respiration stimulation signals from the laryngeal pacemaker 301 to adjacent target neural tissue (e.g., RLN) for vocal fold abduction during respiration of the patient. The respiration sensor 300 is configured to produce a sensed respiration signal for the laryngeal pacemaker 301. The respiration sensor 300 also is configured for transfer and release by guided placement from a sensor applicator to a fixed skin location at the angulus sterni. The laryngeal pacemaker 301 interprets the body motion signal and the sensed respiration signal to make a real time determination of respiratory phase and frequency for adaptively adjusting the respiration stimulation signals accordingly.

A triaxial accelerometer may be integrated into the respiration sensor 300 or the laryngeal pacemaker 301 to produce a body motion signal for the laryngeal pacemaker 301 that reflects energy expenditure of the patient. The body motion signal also can be used for optimizing battery consumption, and the attending body activity detection can be used to determine steady or low movement situations when the laryngeal pacemaker 301 can switch to a paced stimulation to save power.

The laryngeal pacemaker 301 may be configured to compute Energy Expenditure (EE) as a function of x-, y- and z-axis acceleration signals. The tri-axial acceleration body motion signal reflects both a gravitational component and a body motion component, and so the laryngeal pacemaker 301 may need to initially filter and process the body motion signal to extrapolate only the body motion information. Then a Signal Vector Magnitude (SVM) can be computed and compared to pre-determined thresholds that correspond to different body activities. This activity determination can be used to adjust the parameters of the adaptive filtering and peak detection of the bio-impedance signal explained below.

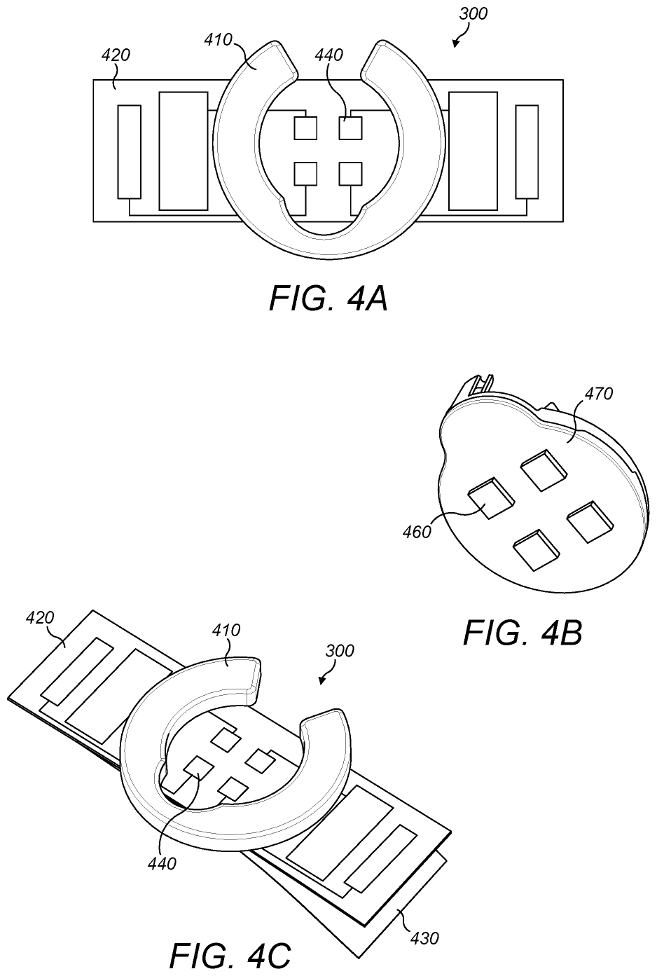

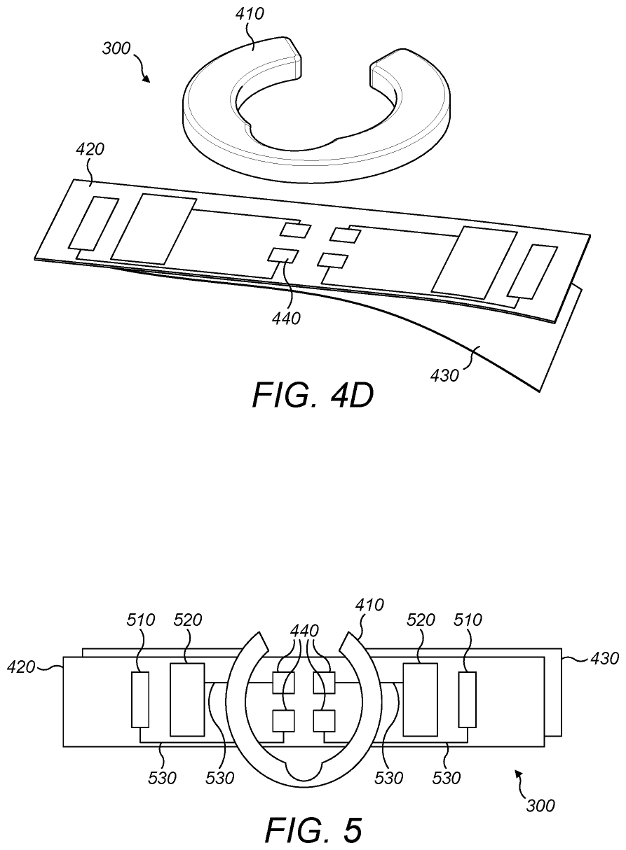

FIGS. 4A-4D and 5 illustrate structural details of such an arrangement. The respiration sensor 300 includes a center support ring 410 that is configured to mechanically engage the respiration sensor 300 with the laryngeal pacemaker 301. The specific shape of the support ring 410 can vary to accommodate different specific design features of the housing of the laryngeal pacemaker 301. Enclosed with the support ring 410 are one or more sensor contacts 440 that are configured to provide a direct electrical connection to corresponding sensor contacts 460 on the bottom surface 470 of the housing of the laryngeal pacemaker 301 when it is enclosed within the support ring 410.

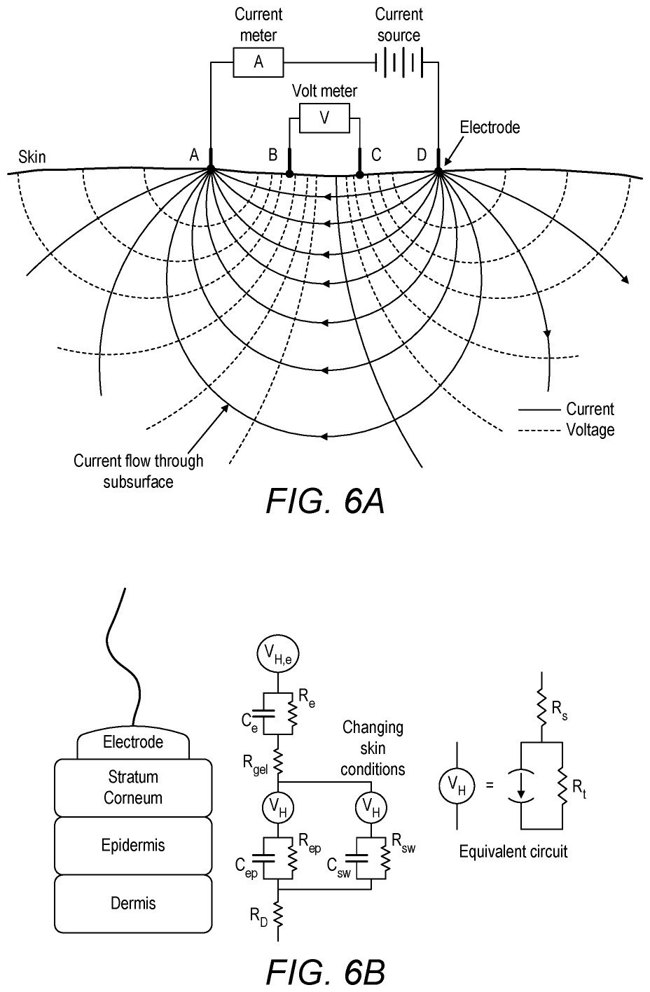

The respiration sensor 300 includes inner sensing contacts 520 and outer excitation contacts 510 that are connected to the sensor contacts 440 by electrically isolated conductive paths 530 made e.g. of gold or other conductive material. The inner sensing contacts 520 and outer excitation contacts 510 are arranged in a tetrapolar configuration for impedance pneumography (IPG) measurement of changes in transthoracic electrical impedance as shown in FIG. 6A. Excitation current flows from the outer excitation contacts 510 (A to D), whereas inner sensing contacts 520 (B and C) measure the corresponding difference of voltage. Changes in the sensed voltage reflect impedance changes in the measured tissue region, where the impedance value Z can be obtained from:

$$Z = \int \frac{1}{p} {J}_{L E} {J}_{LI} dv$$

where ρ is conductivity distribution within the volume conductor ν, J_{LE} is the lead current density field of voltage measurement, J_{LI} is the current density field raised by current injection.

The electrode-skin interface implicates various considerations with regard to recording biological signals. These include the fact that high skin impedance can result in poor signal detection. In addition, relative movement between the electrode and the skin produces motion artifacts. Motion artifacts result from a change in electrical properties of the skin-electrode interface as shown in FIG. 6B. The so-called half-cell potential VH (which results from the charge of the metal-electrolyte interface) can be modelled as a current source and parallel resistor Rt. Resistor Rs represents the stratum corneum, which is an outer skin dielectric layer that decreases the quality of the acquired bio-signal. The half-cell potential VH arises because the current I flows through the resistive extracellular medium Rt. Motion artifacts therefore appear as a potential change due to the current I flowing through the changing resistance Rt which can increase or decrease depending on the nature of the force applied. The relative movement of the electrode with respect to skin can further change the voltage VH. Filtering out and/or reducing motion artifacts is very important.

Wet gel electrodes are commonly used to improve or stabilize the sensing contact and reduce skin impedance by increasing the conductive of the stratum corneum layer. Any mechanical disturbances caused by relative motion between the electrode and the skin are damped by the intervening gel layer, and their effect on the signal is limited. They can be schematized as almost resistive impedance, whose value is in the range of few decades of Ohms. The equivalent impedance Zequi derived from FIG. 6B therefore can be expressed as:

Z_{equi}=Rₑ∥Cₑ+R_{gel}+Rₛ+Rₜ+Rₑₚᵢ∥Cₑₚᵢ+R_{d}

where Rₑ, Cₑ and R_{gel} all depend on the specific type of electrode and its coupling with the skin. They can change during body movement and still create motion artifacts, although the changed value is reduced as long as the wetting gel does not dry off. When the gel does dry off, the value of R_{gel} increases and the coupling with the skin dramatically decreases. Therefore, long term measurements (i.e. experiments over more consecutive days) are not possible when using standard gel electrodes.

That issue can be addressed if the respiration sensor 300 is made from ultrathin and ultra-conformable flexible nanosheets composed of conducting polymer complex poly (PEDOT:PSS) that form a printed tattoo electrode 420 which provide ultra-conformability on a complex surface such as skin. Using such a material, the respiration sensor 300 specifically may form Tattoo Conductive Polymer Nanosheets for Skin-Contact Applications temporary printed tattoos that are transferred and released to the skin location, thereby overcoming the issue with lack of conformability and poor adhesion that usually occurs with standard dry electrodes.

On the underside of the respiration sensor 300 is an electrode liner 430 that is removed when applying the respiration sensor 300 to the skin. When the electrode liner 430 is removed, the respiration sensor 300 is released to the skin by gently and uniformly rubbing a wet finger (or any other equivalent means) over the top surface of the respiration sensor 300. The consequent release of the respiration sensor 300 to the skin will occur in a few seconds.

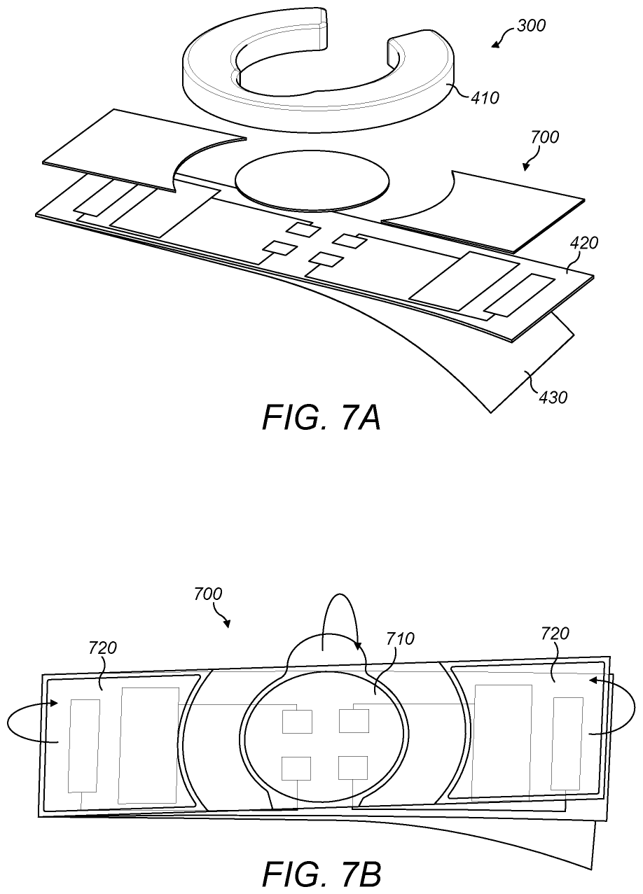

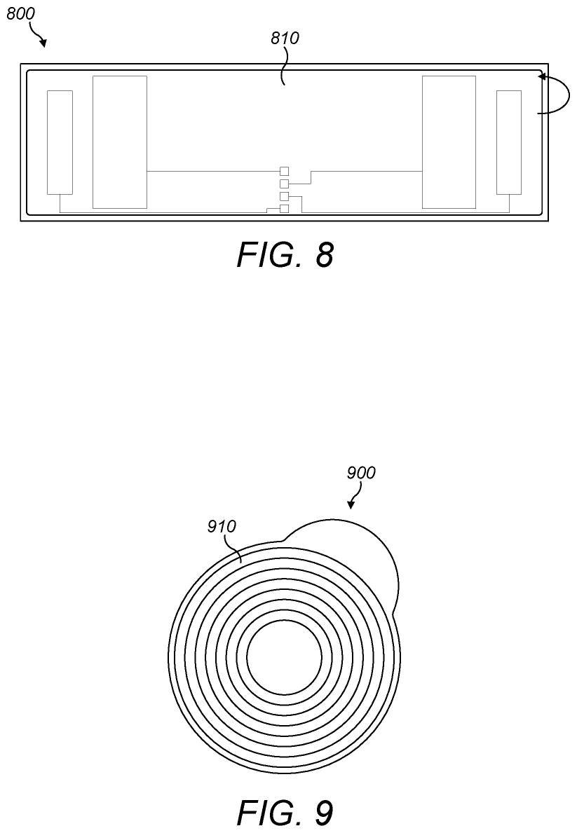

In the embodiment of a respiration sensor 300 shown in FIGS. 7A-7B, there is an additional semi-rigid support layer 700 that lies between the support ring 410 and the printed tattoo electrode 420 that is configured to provide mechanical support to the printed tattoo electrode 420. The support layer 700 includes an inner water pocket 710 and two outer water pockets 720 that form an integrated water release system to release a wetting layer of water when mechanically pressed to further promote transfer of the printed tattoo electrode 420 to the skin. The water pockets 710 and 720 are configured to break and release water when finger pressure is applied. After the water has been uniformly released to the underlying printed tattoo electrode 420, the support layer 700 can be torn off as shown by the arrows. While the support layer shown in FIGS. 7A-7B includes a plurality of water pockets, an embodiment as shown in FIG. 8 could have a support layer 800 with a single water pocket 810.

FIG. 9 shows the bottom surface 900 of a laryngeal pacemaker with sensor contacts 910 in the form of multiple concentric rings. This arrangement would allow the laryngeal pacemaker to freely rotate about its axis while still be able to acquire the sensed respiration signal.

The laryngeal pacemaker 301 performs adaptive filtering processing of the bio-impedance sensed respiration signal from the respiration sensor 300 in combination with the body motion activity determination from the accelerometer signal. The sensed respiration signal typically is defined in the range [0.1 Hz-1 Hz] in dependence on the body activity occurring. Thus the adaptive filtering of the sensed respiration signal is directed achieving the greatest possible Signal to Noise Ratio (SNR) over the different real-life situations that the patient may encounter. Since the motion artifact noise that affects the sensed respiration signal cannot be considered Gaussian, the adaptive filtering may be based on a Least Mean Square Filter (LMS) approach. By definition, the convergence of the error between the actual and desired signal cannot be guaranteed with LMS (as compared to approaches based on a Kalman filter) and only depends on the number of the iterations performed.

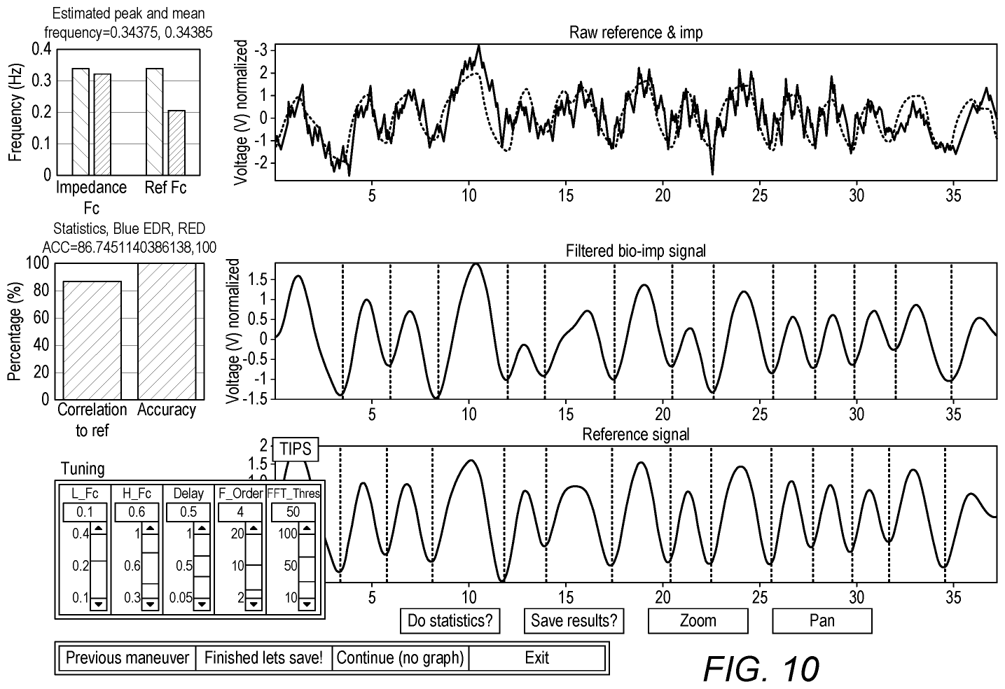

Once the algorithm has been initialized (i.e. learning rate, number of iterations and delay), the sensed respiration signal can be filtered in real time without any further external input. The filtered sensed respiration signal is then used to detect the onset of the respiration as shown in FIG. 10. The first row waveform in FIG. 10 shows the raw bio-impedance and reference signals overlapped. The reference signal is acquired by a thermistor that the patient wears during the acquisition together with the sensor and electrodes measuring the bio-impedance. The second and third row waveforms show the filtered bio-impedance and reference signal respectively. The respiration onsets of the signals are shown by the vertical lines on the waveforms. Accuracy detection is computed together with signal correlation in the histogram plots on the left side of FIG. 10 (100% and 94% respectively for this particular experiment). Respiration onset detection is considered successful when the onset in the bio-impedance signal is detected within a maximum delay of 500 msec from the true onset shown in the reference signal. A Bayesian peak validation algorithm can later be used to identify the true peaks based on a Kalman filter.

Embodiments of a laryngeal pacemaker and respiration sensor as described above allow for real time stimulation signals to the target neural tissue that can be triggered by the real time respiratory signal rather than on the base of pre-determined pacing. In addition, the described respiration sensor is not invasive and can be embedded/integrated into the housing of the laryngeal pacemaker without increasing its external dimensions. Moreover, long term monitoring can be provided since the set up the printed tattoo electrode overcomes the issues of wet gel electrodes and can provide ultra-conformability and adhesion on the skin for up to three days. The extremely reduced thickness of the tattoo electrode and the absence of adhesive material also provides for increased comfort and wearability of the respiration sensor. And the tetrapolar electrode configuration together with the placement on the chest at the angulus sterni provides increased robustness to motion artifacts.

The respiration sensor technology described above that is based on Printed Tattoo Electrodes (PTE) can provide ultra-conformability on a complex surface like skin. Their transfer and release as temporary tattoos overcomes the issue with lack of conformability and poor adhesion which usually occurs with standard dry electrodes. And the general field of flexible and skin-transferable sensors is itself receiving ever greater interest due to the potential of developing highly integrated sensors for monitoring vital signs that are portable and not overly invasive.

However, one limiting factor preventing more widespread use of this technology lies in the complexity of the electrode release process. It is difficult to provide a uniform pre-determined release of water onto the tattoo surface, and that together with the need for extra-care in handling such a soft and floppy material often requires an expertly trained subject to facilitate the transfer and release process. The sensor has to be pressed onto the skin strongly enough to provide adequate contact, but not too strongly so as to prevent breakage. In addition, a replacement sensor needs to be applied in the same way as the previously removed one, in the preferred horizontal position in order to properly attach to the skin on both sides of the angulus sterni.

Thus, embodiments of the present invention include a sensor applicator configured to provide reliable, easy and effective placement and release of the sensor by means of a user-friendly design that avoids mis-application. The transfer and release of the PTE sensor can be performed by the patients themselves without needing additional assistance of a trained expert. The sensor applicator guides the patient to correctly place the PTE sensor in the desired correspondence with the angulus sterni in an exactly horizontal positioning, and the sensor applicator can provide feedback to the patient on whether or not the transfer and release process occurred correctly. Embodiments of such a sensor applicator can be used for the placement of any flexible and skin-transferable sensor.

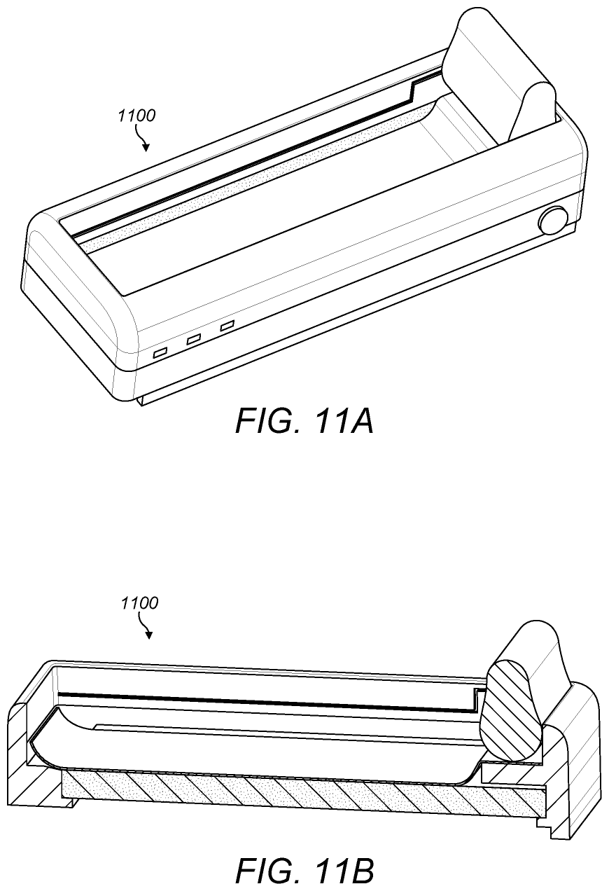

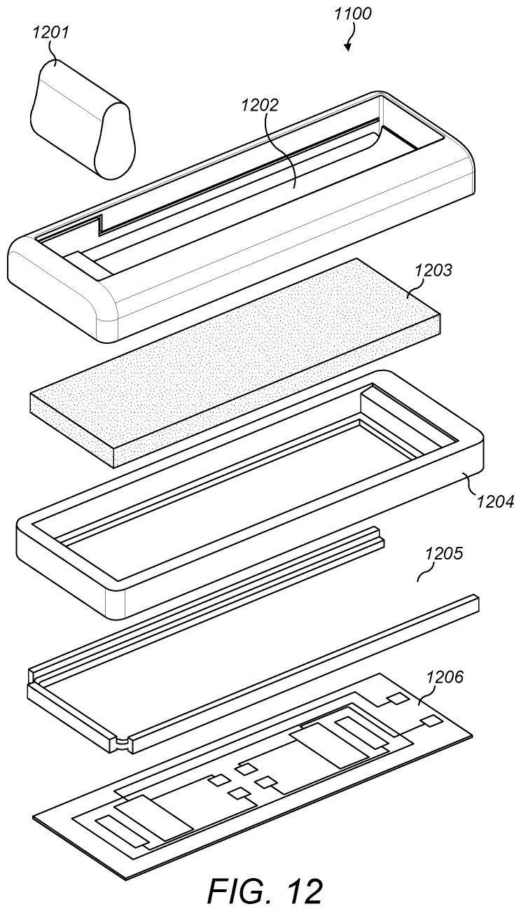

FIGS. 11A-11B show different views of a sensor applicator 1100 according to an embodiment of the present invention. Specific sensor applicator arrangements may make use of active electronics, passive electronics only, or no electronics at all. FIG. 12 shows an exploded view of different structural elements the sensor applicator 1100. A pressure roller 1201 fits and slides within an upper housing 1202. Lower housing frame 1204 and bottom surface 1205 fit together to form a holding receptacle configured to contain a fluid storage sponge 1203. For example, fluid storage sponge 1203 may be made of sponge material or any other equivalent material that can absorb and hold a small amount of release fluid such as water. The PTE sensor 1206 is inserted into the bottom of the sensor applicator 1100.

Pressure roller 1201 fits within two sliding tracks located within the upper housing 1202 to slide freely along the longitudinal axis of the upper housing 1202. The bottom side of the upper housing 1202 is a flexible pressure surface on top of which the pressure roller 1201 can be pushed along its displacement trajectory. In specific embodiments, the pressure surface of the upper housing 1202 may be a simple water resistant foil, or in more advanced embodiments, it may be made of a piezo-chromic material that exhibits a reversible color change under pressure effect. Reversible piezo-chromic materials with memory-effect change color at some initial activation pressure P1, and then recover the original color when the applied pressure drops below second recovery pressure P2. The difference between these two pressures P1 and P2 defines the memory effect and allows specification of the history of the material (i.e. if the material has exceeded a threshold of pressure). Once the activation pressure P1 needed for an optimal release of the PTE sensor 1206 onto the skin has been applied using the pressure roller 1201, the color change of the piezo-chromatic material of the bottom surface can provide user feedback on whether the applied pressure was sufficient and/or uniform enough to guarantee a successful release (or not).

Besides the color-based pressure surface release feedback arrangement described above, there are other ways to provide user feedback about the release process. For example, the upper housing 1202 and/or the lower housing frame 1204 may incorporate signal acquisition sensors configured to receive the sensed ECG signal from the released PTE sensor 1206 when it is applied to the patient's skin. Simple electronics within the sensor applicator 1100 then can perform a signal level thresholding and determine whether or not an acceptable signal has been detected. If acceptable, a feedback LED on the body of the sensor applicator 1100 turns green to confirm the successful release of the PTE sensor 1206.

Lower housing frame 1204 and bottom surface 1205 fit may be structurally separate pieces configured to fit together, or they may be integrated together into a single structural element. In either case, the bottom surface 1205 is configured to securely hold the PTE sensor 1206 while it is within the sensor applicator 1100. For example, the bottom surface 1205 may incorporate one or more locking clips arranged to move cooperatively or independently, for example, along a perpendicular axis of the bottom surface 1205. Such locking clips are configured to provide appropriate support to avoid the PTE sensor 1206 inadvertently slipping away from the bottom surface 1205. For example, the locking clips can have a saw tooth profile that enhances the security of the PTE sensor 1206 to the bottom surface 1205.

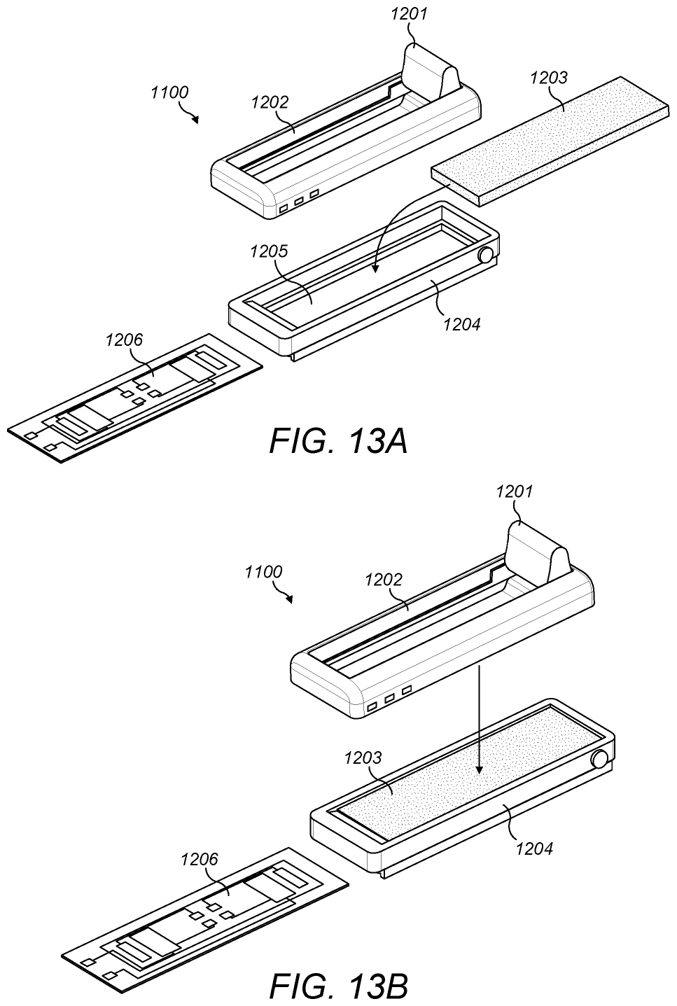

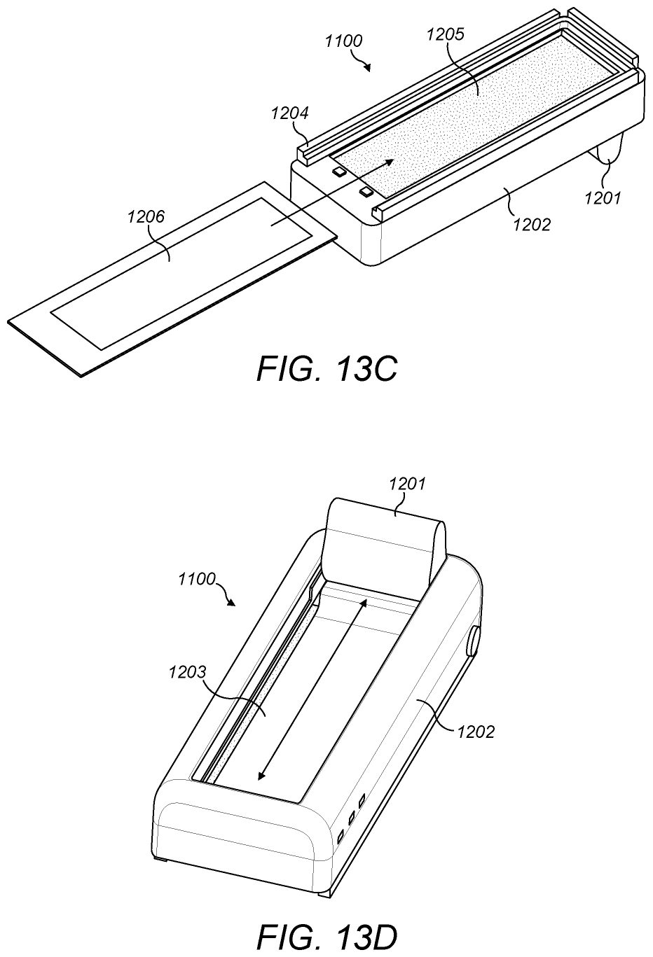

FIGS. 13A-13D show various aspects in using a sensor applicator according to an embodiment of the present invention. Initially, as shown in FIG. 13A, the sensor applicator 1100 is disassembled so that the fluid storage sponge 1203 needs to be inserted into the lower housing frame 1204 as shown by the thick arrow, and then, as shown by FIG. 13B, the upper housing 1202 lower housing frame 1204 are coupled together. The sensor applicator 1100 can then be inverted and the PTE sensor 1206 can be inserted into the lower housing frame 1204 against the bottom surface 1205 as shown in FIG. 13C. At this point, the electrode liner 430 is removed. Finally, the assembled sensor applicator 1100, as shown in FIG. 13D, is ready to be placed on the chest of the patient.

Correct placement of the assembled sensor applicator 1100 at the angulus sterni can be confirmed by one or more LEDs in the side of the sensor applicator 1100. For example, a magnetic sensor within the sensor applicator 1100 can be configured to detect proximity of the device to the implanted magnet at the angulus sterni location, and first positioning LED would then turn green. Moreover, the PTE sensor needs to be placed as closely as possible to horizontal. For that purpose, the sensor applicator 1100 may also contain a sensing gyroscope configured to detect when the device is positioned horizontally within some pre-determined angle of tolerance, and a second positioning LED would then turn green confirming the horizontal position.

At this point, the patient is holding the loaded sensor applicator 1100 on the chest with one hand, and the other hand can then move the pressure roller 1201 within the sensor applicator 1100 to activate the water-based release mechanism. In some specific embodiments, the release mechanism may be further promoted if the patient also pushes down on the pressure roller 1201. Activation of the pressure roller 1201 squeezes the fluid storage sponge 1203 within the sensor applicator 1100, causing a uniform water release onto the PTE sensor 1206 that transfers it to the skin. Depending on the specific release feedback mechanism (as discussed above) the patient gets release feedback on whether or not the release movement needs to be repeated. For example, the confirming color change of the pressure surface of the upper housing 1202 or a third release LED turning green confirms a correct conclusion of the sensor application process.

As explained above, specific embodiments of the sensor applicator 1100 may incorporate active electronic elements such as a magnetic sensor, a gyroscope, and/or sensor signal threshold detection logic that are configured to support the patient in the correct placement of the device on the chest. However, specific embodiments of the sensor applicator 1100 may in addition or alternative use passive elements for one or more of the same purposes. For example, the proximity of the sensor applicator 1100 to the implanted magnet may be detected by placing another magnet within the applicator at its center. Horizontal placement of the sensor applicator 1100 may be achieved by using a spirit level in the longitudinal side of the device. In addition, the piezo-chromatic effect described above for release feedback already represents a passive approach.

Embodiments of a sensor applicator as described above reflect a user-friendly design that is easy and intuitive to use. This promotes the precision and reliability of the transfer and release of the sensor device by the patients themselves without the need of an additional expert. Moreover, a sensor applicator with a sponge-based water release mechanism may be even better performing than the breakable water pocket solutions described earlier. In addition, a respiration that is applied by a sensor applicator as just described may avoid the need for incorporating a support ring into the sensor device. That would reduce the cost per unit of the sensor device.

Embodiments of the invention may be implemented in part in any conventional computer programming language such as VHDL, SystemC, Verilog, ASM, etc. Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A laryngeal pacing system for a recipient patient with impaired breathing, the system comprising:
a laryngeal pacemaker configured for external placement on skin of a patient at a sternum location and configured to produce respiration stimulation signals;
an implantable stimulation electrode configured for delivering the respiration stimulation signals from the laryngeal pacemaker to adjacent target neural tissue for vocal fold abduction during respiration of the recipient patient;
a triaxial accelerometer configured to produce a body motion signal for the laryngeal pacemaker reflecting energy expenditure of the recipient patient; and
a respiration sensor comprising a flexible skin-transferrable printed tattoo electrode having a tetrapolar configuration for impedance pneumography measurement to produce a sensed respiration signal for the laryngeal pacemaker, the respiration sensor, configured for transfer and release using a water-based transfer mechanism, further comprising a semi-rigid support layer configured to provide mechanical support to the printed tattoo electrode and configured to release a wetting layer of water when mechanically pressed;
wherein the respiration sensor is configured for transfer and release by guided placement from a sensor applicator to a fixed skin location at the angulus sterni of the recipient patient; and
wherein the laryngeal pacemaker is configured to interpret the body motion signal and the sensed respiration signal to make a real time determination of respiratory phase and frequency for adaptively adjusting the respiration stimulation signals accordingly.

2. The laryngeal pacing system according to claim 1, wherein the laryngeal pacemaker includes an outer surface having a plurality of sensor contacts configured to directly connect to the respiration sensor for coupling the sensed respiration signal from the respiration sensor to the laryngeal pacemaker.

3. The laryngeal pacing system according to claim 1, wherein the triaxial accelerometer is integrated into the laryngeal pacemaker.

4. The laryngeal pacing system according to claim 1, wherein the printed tattoo electrode comprises tattoo conductive polymer nanosheets for skin-contact applications.

5. The laryngeal pacing system according to claim 1, wherein the respiration sensor further comprises a center support ring configured to mechanically engage the respiration sensor with the laryngeal pacemaker.

6. The laryngeal pacing system according to claim 1, wherein the support layer includes a plurality of water-holding sub-divisions.

7. The laryngeal pacing system according to claim 1, wherein the support layer includes a single water-holding sub-division.

8. The laryngeal pacing system according to claim 1, wherein the respiration sensor is adapted to cooperate with the sensor applicator to provide positioning feedback information when the respiration sensor is placed at the fixed skin location.
